(19) United States
(12) Patent Application Publication
(10) Pub. No.: US 2008/0039784 A1
(43) Pub. Date: Feb. 14, 2008

(54) Multi-Lumen Catheter System

(75) Inventors: David Quinn, Galway (IE); Ashish Varma, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/877,222

(22) Filed: Oct. 23, 2007

Related U.S. Application Data
(63) Continuation of application No. 10/805,518, filed on Mar. 22, 2004.

(51) Int. Cl. A61M 25/10

(52) U.S. Cl. 604/96.01; 604/526

(57) ABSTRACT

A multi-lumen catheter with a generally circular cross-section having a central guidewire lumen, partially circumscribed by an inflation lumen having a generally C-shaped cross-section defining a web and a guidewire access cut extending radially through that web from the outer surface of the multi-lumen catheter to the central guidewire lumen. The guidewire access cut allowing access to an indwelling guidewire for direct control over axial translation, or ingress and egress of the guidewire.

### CROSS-REFERENCE TO RELATED APPLICATION

This application is a continuation of pending U.S. application Ser. No. 10/805,518, filed Mar. 22, 2004, which is hereby incorporated by reference in its entirety.

### FIELD OF THE INVENTION

The present invention relates to multi-lumen catheters used with guidewires and, in particular, to a system facilitating control over the guidewire independent of the multi-lumen catheter.

### BACKGROUND OF THE INVENTION

Cardiovascular disease, including atherosclerosis, is a leading cause of death in the U.S. The medical community has responded by developing a number of methods and devices for treating coronary heart disease. Some of those methods and devices are specifically designed to treat the complications resulting from atherosclerosis and other forms of coronary arterial narrowing.

One method for treating atherosclerosis, in addition to other forms of coronary narrowing, is percutaneous transluminal coronary angioplasty, commonly referred to as “angioplasty” or “PTCA”. The objective in angioplasty is to enlarge the lumen of the affected coronary artery by hydraulically expanding a device placed within the affected body lumen. The procedure is commonly performed by inflating the balloon of a balloon catheter within the narrowed region of the coronary artery.

Catheters have become utilized in many procedures beyond treating coronary heart disease. For example, they are used for delivery of stents, grafts, therapeutic substances (such as anti-vaso-occlusion agents or tumor treatment drugs) and radiopaque agents for radiographic viewing.

The anatomy of coronary arteries varies widely from patient to patient. Often a patient's coronary arteries are irregularly shaped, highly tortuous and very narrow. The tortuous configuration of the arteries may present difficulties to the physician in proper placement of a guidewire, and advancement of a catheter to a treatment site. A highly tortuous coronary anatomy typically will present considerable resistance to advancement of the catheter over the guidewire.

Therefore, it is important for a catheter to be highly flexible. However, it is also important for a catheter shaft to be stiff enough to progress the catheter through the vessel in a controlled manner from a position far away from the distal end of the catheter.

Conventional catheter shafts for PTCA and other procedures typically include a proximal shaft, a transition section and a distal shaft terminating at a flexible tip. Generally, the proximal shaft is relatively rigid to allow for increased pushability and has a guidewire lumen extending throughout its length. In contrast, the distal shaft is generally a flexible polyethylene sleeve with a flexible polyethylene tube disposed concentrically within the sleeve and extending from the guidewire lumen at the distal end of the proximal shaft, through the transition section and the distal shaft. Typically, the distal shaft extends for a length on the order of 25 centimeters and allows for curving through particularly tortuous vessels. The transition section provides a gradual transition in stiffness between the relatively stiff proximal shaft and the flexible distal shaft. Including the transition section reduces the tendency of portions of the catheter, particularly where the rigid proximal shaft and the flexible distal shaft meet, to collapse, buckle or kink.

In a typical PTCA procedure, it may be necessary to perform multiple dilatations, for example, using various sized balloons. In order to accomplish the multiple dilatations, the original catheter must be removed and a second catheter tracked to the treatment site. When catheter exchange is desired, it is advantageous to leave the guidewire in place while the first catheter is removed to properly track the second catheter.

Two types of catheters commonly used in angioplasty procedures are referred to as over-the-wire (OTW) catheters and rapid exchange (RX) catheters. A third type of catheter with preferred features of both OTW and RX catheters, which is sold under the trademarks MULTI-EXCHANGE, ZIPPER MX, ZIPPER, MX and/or MXII, is discussed below. An OTW catheter's guidewire lumen runs the entire length of the catheter and the entire length of an OTW catheter is tracked over a guidewire during a PTCA procedure. A RX catheter, on the other hand, has a guidewire lumen that extends within only the distalmost portion of the catheter. Thus, during a PTCA procedure only the distalmost portion of a RX catheter is tracked over a guidewire.

If a catheter exchange is required while using a standard OTW catheter, the user must add an extension onto the proximal end of the guidewire to maintain control of the guidewire during the exchange and to maintain its sterility. Once the extension is added, the clinician can slide the catheter off of the extended guidewire, slide the new catheter onto the guidewire and track the new catheter to the original catheter position. Due to the length of the extended guidewire, multiple operators are required to hold the extended guidewire in place while the original catheter is removed.

A RX catheter avoids the need for multiple operators when changing catheters. With a rapid exchange catheter, the majority of the guidewire resides outside of the catheter. The guidewire enters the catheter only in the distalmost portion. That exposure of the guidewire allows it to be held in place when the catheter is removed from the body without necessitating the addition of a guidewire extension. Although the guidewire exposure simplifies catheter exchange, it can create a problem with entanglement between the exposed portion of the guidewire and the catheter shaft during use.

There are other instances when the guidewire must be replaced and the catheter left indwelling. An OTW catheter, with the guidewire lumen extending the entire length of the catheter, allows for simple guidewire exchange. A rapid exchange catheter, on the other hand, is not so accommodating. To replace a guidewire with a RX catheter, the guidewire and most of the catheter must be removed from the body. Essentially, the procedure must then start anew because both the guidewire and the catheter must be returned to the treatment site.

A balloon catheter capable of both fast and simple guidewire and catheter exchange is particularly advantageous. A catheter designed to address this need is sold by Medtronic Vascular, Inc. of Santa Rosa, Calif. under the trademarks MULTI-EXCHANGE, ZIPPER MX, ZIPPER, MX and/or MXII (hereinafter referred to as the “MX catheter”). An MX catheter is disclosed in U.S. Pat. No. 4,988,356 to Crittenden et al.; U.S. Pat. No. 6,800,065; U.S. Pat. Appl. Publ. No. 2004/0059369; U.S. Pat. No. 6,905,477; U.S. Pat. Appl. Publ. No. 2004/0260329 A1; and U.S. Pat. No. 6,893,417, all of which are incorporated by reference in their entirety herein.

The MX catheter includes a proximal catheter shaft having a guidewire lumen positioned side-by-side with an inflation lumen. The MX catheter also includes a longitudinal cut that extends along the proximal catheter shaft and that extends radially from the guidewire lumen to an exterior surface of the proximal catheter shaft. A guide member that is slideably coupled with the proximal shaft cooperates with the longitudinal cut such that a guidewire may extend transversely into or out of the guidewire lumen at any location along the longitudinal cut's length. By moving the shaft with respect to the guide member, the effective over-the-wire length of the MX catheter is adjustable.

In the MX catheter, a guidewire is threaded into a guidewire lumen through an opening at the distal end of the catheter and out through the guide member. The proximal guidewire lumen envelops the guidewire as the catheter is advanced into the patient's vasculature. Furthermore, the indwelling catheter may be removed by withdrawing the catheter from the patient while holding the proximal end of the guidewire and the guide member in a fixed position. When the catheter has been withdrawn to the point where the distal end of the cut has reached the guide member, the distal portion of the catheter over the guidewire is of a sufficiently short length that the catheter may be drawn over the proximal end of the guidewire without releasing control of the guidewire or disturbing its position within the patient.

In order to accommodate an inflation lumen and a guidewire lumen disposed in a side-by-side relationship in the proximal catheter shaft, the catheter shaft may be made with an oblong or oval shaped cross-section. Although such a cross-section provides good pushability and trackability through a patient's vasculature, some clinicians who are accustomed to circular shafts find the feel of such shafts uncomfortable. In addition, it is easier to provide a better balance between back-bleed and interaction with a Touhy Borst fitting with a circular shaft which would lead to a reduction in friction between the catheter and the fitting. Thus, it is an object of this invention to provide the benefits of an MX catheter with a proximal catheter shaft having a side-by-side lumen relationship with an overall circular cross-section.

### BRIEF SUMMARY OF THE INVENTION

The present invention is a proximal catheter shaft constructed from an elongate tubular body with a generally circular cross-section that provides multiple lumens extending longitudinally throughout the length. The lumens include a central guidewire lumen and a peripheral inflation lumen that circumscribes the guidewire lumen. The inflation lumen has a generally C-shaped, or a partial annulus, cross-section. The discontinuous annulus shape of the inflation lumen defines a web and through that web extends a guidewire access cut.

The catheter shaft may rely upon an indwelling guidewire for stiffness or it may employ additional stiffening elements. When additional stiffening elements are included, they may include metal or polymer inserts extruded into the wall of the catheter shaft between the lumens. Alternatively, stiffening elements may be incorporated into a lumen. Furthermore, additional lumens may be included in the tubular body specifically designed to hold a fluid, thereby increasing the stiffness of the shaft. The stiffening members may be further customized to create a region where the shaft transitions from a relatively high stiffness to a relatively low stiffness.

The guidewire access cut extends radially through the web from the outer surface of the catheter to the guidewire lumen and provides direct access to the guidewire for a guidewire control member slideably mounted to the catheter. The guidewire control member may provide direct axial control over movement of the guidewire relative to the catheter shaft, or alternatively, may provide a means for ingress and egress of the guidewire from the guidewire lumen.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art based on the teachings contained herein.

### BRIEF DESCRIPTION OF THE DRAWINGS

These and other features, aspects and advantages of the present invention will become better understood with reference to the following description, appended claims, and accompanying drawings where:

FIG. 1 is a side elevational view of the multi-lumen catheter incorporating the present invention.

FIG. 2 is a cross-sectional view of the multi-lumen catheter of FIG. 1 taken along line A-A.

FIGS. 3A-3F illustrate various embodiments of stiffening members integrated into the multi-lumen catheter of FIG. 1 shown in a cross-sectional view taken along line A-A.

FIGS. 4A-4B illustrate various embodiments of stiffening member transitional sections.

FIG. 5 is a side elevational view of a first embodiment of the guidewire control member.

FIG. 6 is a cross-sectional view of the guidewire control member of FIG. 5 taken along line B-B.

FIG. 7 is a cross-sectional view of the guidewire control member of FIG. 5 taken along line C-C.

FIG. 8 is a side elevational view of a second embodiment of the guidewire control member.

FIG. 9 is a side elevational view of the outer tubular member of the guidewire control member of FIG. 8.

FIG. 10 is side elevational view of the inner body of the guidewire control member of FIG. 8.

FIG. 11 is a cross-sectional view of the inner body of FIG. 10 taken along line D-D.

FIG. 12 is a side elevational view of a third embodiment of the guidewire control member.

FIG. 13 is a cross-sectional view of the guidewire control member of FIG. 12 taken along line E-E.

FIG. 14 is a cross-sectional view of the guidewire control member of FIG. 12 taken along line F-F.

### DETAILED DESCRIPTION OF THE INVENTION

The present invention is now described with reference to the figures where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention.

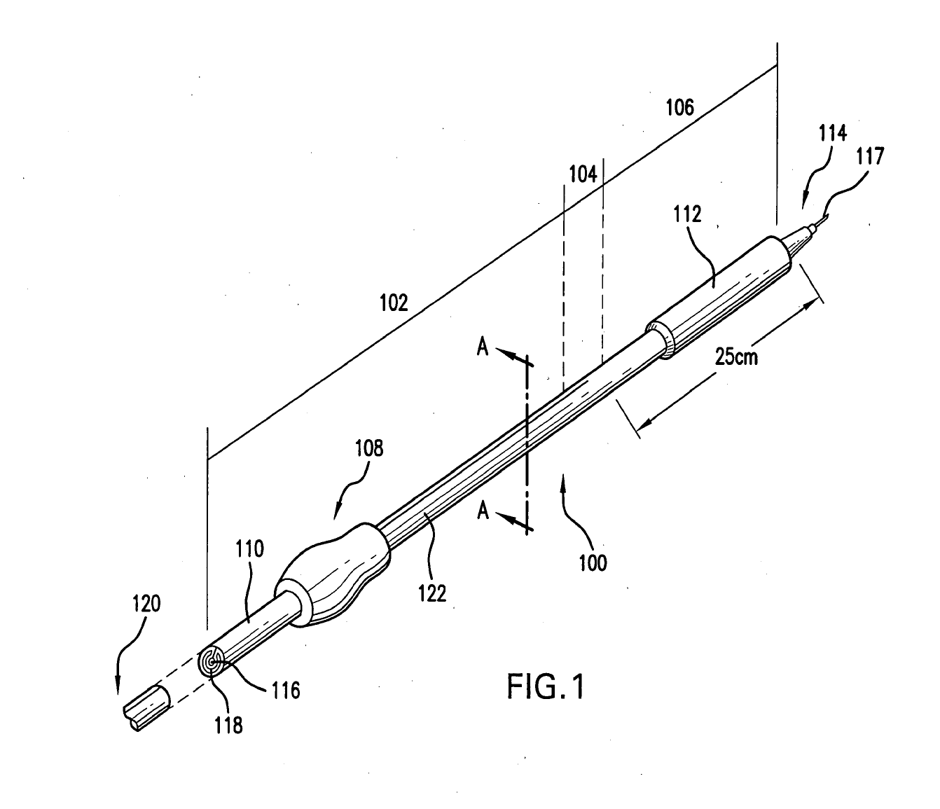

As shown in the exemplary embodiment of FIG. 1, the invention includes a multi-lumen catheter, indicated generally by reference numeral 100, having a proximal shaft 102 on which a guidewire control member 108 is slideably mounted, a transition section 104 and a distal shaft 106. A guidewire 117 is shown extending out of a distal tip 114 of multi-lumen catheter 100. Guidewire 117 is slidably received within a guidewire lumen 116. Guidewire control member 108 slides longitudinally along the periphery of proximal shaft 102 and allows a clinician to independently manipulate guidewire 117 and multi-lumen catheter 100 while not interfering with an inflation lumen 118. It shall be appreciated that guidewire control member 108 generally allows the clinician independent control of guidewire 117 and multi-lumen catheter 100 while guidewire control member 108 is located at any point along the length of proximal shaft 102.

In the embodiment shown in FIG. 1, multi-lumen catheter 100 is a balloon catheter, such as for PTCA or stent delivery, having a balloon 112 mounted on a distal portion of the catheter near distal tip 114. Balloon 112 may be inflated and deflated through inflation lumen 118 formed through proximal shaft 102 of multi-lumen catheter 100. Inflation lumen 118 extends from a proximal end 120 of multi-lumen catheter 100 through the length of proximal shaft 102 and transition section 104, terminating in fluid communication with the interior of balloon 112. Proximal shaft 102 also includes guidewire lumen 116 with a variable working length, which is intended to receive guidewire 117.

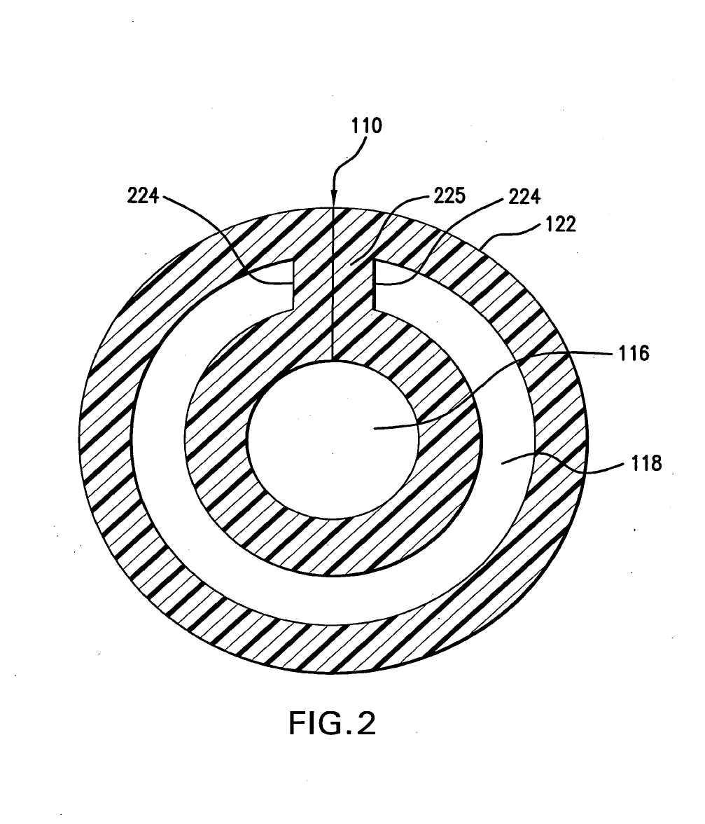

In accordance with the invention, the inflation lumen 118 is disposed generally concentrically about a portion of guidewire lumen 116. Inflation lumen 118 partially circumscribes guidewire lumen 116 resulting in inflation lumen 118 having a generally C-shaped, or partial annulus, cross-section as shown in FIG. 2. The generally C-shaped cross-section of inflation lumen 118 results in two inflation lumen ends 224 wherein the space between those two inflation lumen ends 224 defines a web 225. Web 225 extends radially from guidewire lumen 116 to the outer surface 122 of multi-lumen catheter 100 and is bisected by a guidewire access cut 110. Guidewire access cut 110 may extend the entire length of proximal shaft 102 and may extend into transition section 104. In operation, spreading guidewire access cut 110 provides a thoroughfare for direct access to an indwelling guidewire or to insert or remove a guidewire from guidewire lumen 116.

The far proximal end 120 of the multi-lumen catheter 100 terminates with a hub (not shown). The hub is tailored to the type of guidewire control member 108 employed. Guidewire control member 108 may have one of many forms depending on the required utility. For example, guidewire control member 108 may be used to vary the effective OTW length of the multi-lumen catheter 100 in which case guidewire control member 108 will provide a proximal exit for guidewire 117. As a result, a single lumen hub, such as a Luer fitting, would be used. On the other hand, if the guidewire control member is used solely to assist with manipulation of guidewire 117, a bifurcated hub would be included.

Proximal shaft 102 is an elongate, flexible, tubular shaft which may be formed from polymeric materials, particularly high-density polyethylene, polyimide, polyamides, polyolefins, polyethylene block amide (PEBAX®) copolymer and various other polymeric materials suitable for use in medical devices. Preferably, proximal shaft 102 is made from high-density polyethylene due to its low friction characteristics. Proximal shaft 102 may be extruded or formed in another process known in the art for producing multi-lumen tubing used in a medical device.

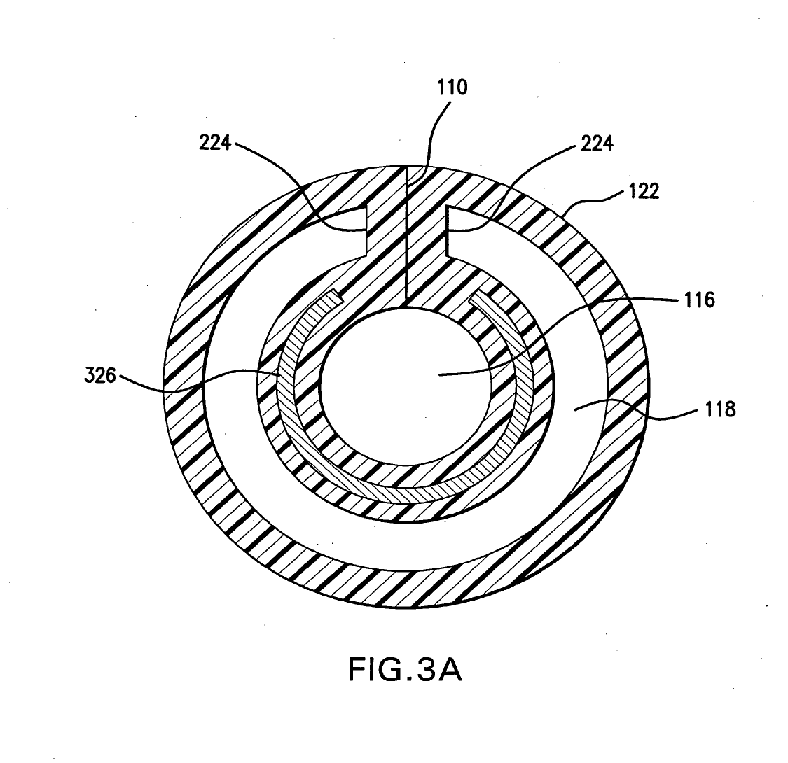

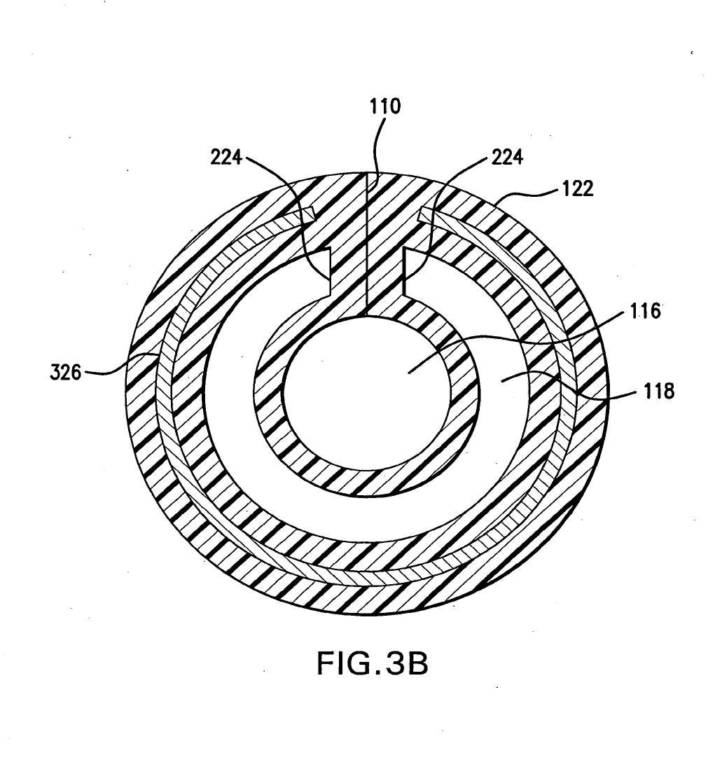

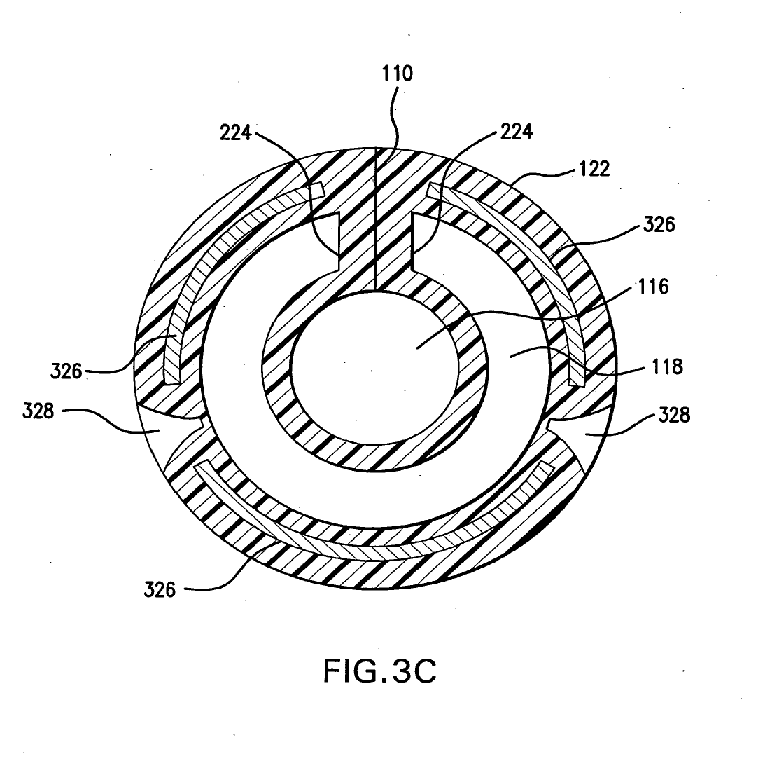

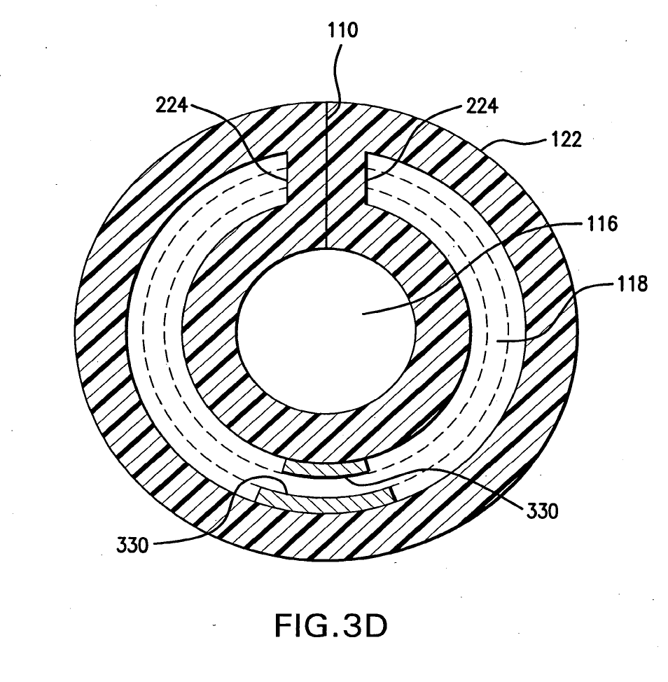

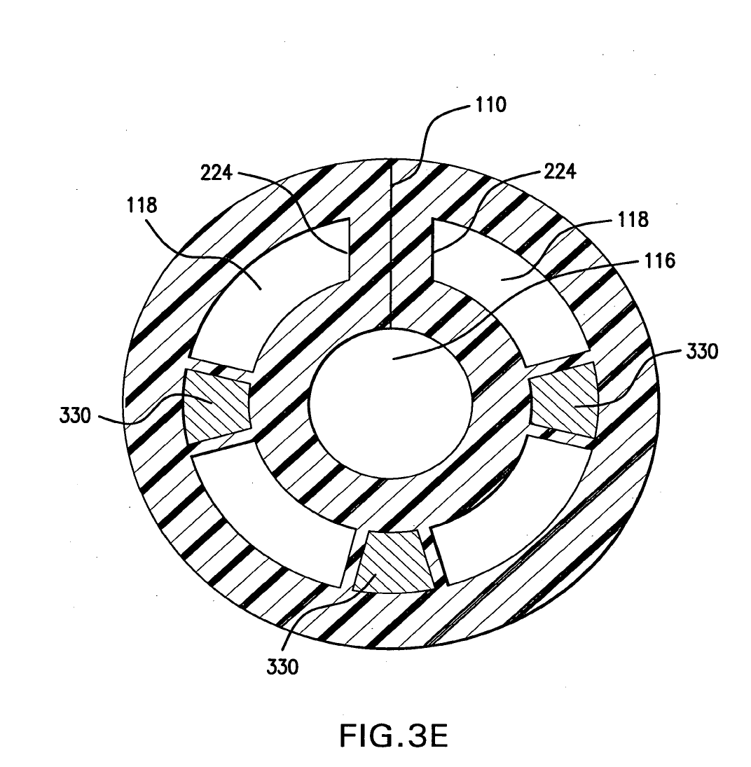

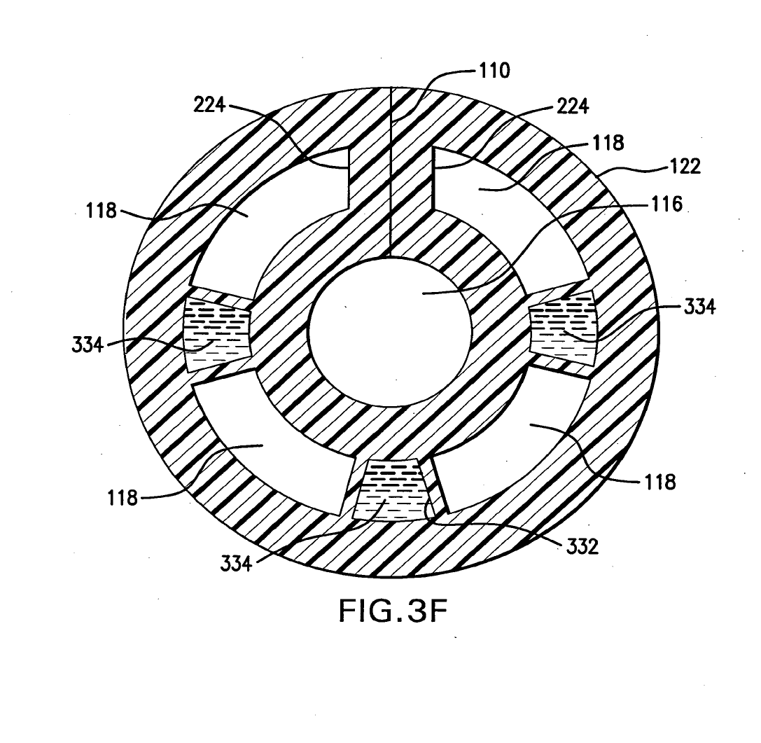

The longitudinal stiffness of proximal shaft 102 may be customized. In the embodiment shown in FIG. 2, the longitudinal stiffness is derived mainly from the longitudinal stiffness of guidewire 117 threaded through guidewire lumen 116. Alternatively, additional stiffening features may be included, as shown in the various embodiments illustrated in FIGS. 3A-3F.

FIG. 3A is a cross-sectional view of one embodiment of proximal shaft 102 with a stiffening member 326 extruded into the catheter shaft. In this embodiment, stiffening member 326 is disposed between guidewire lumen 116 and inflation lumen 118. In another embodiment, stiffening member 326 may be disposed between inflation lumen 118 and outer catheter surface 122 as shown in FIG. 3B.

In a still further embodiment, multiple stiffening members 326 may be extruded into proximal shaft 102 as shown in FIG. 3C. Further still, proximal shaft 102 may also include a joint 328 between stiffening members 326. The inclusion of joints 328 allows greater freedom in the customization of the stiffness of the proximal shaft without hindering the spreading of guidewire access cut 110. Joints 328 may be manufactured as a void or groove in the wall of proximal shaft 102 or a second material may be utilized.

Where a second material is used, proximal shaft 102 may be created by a triple extrusion process wherein a triple extrusion die allows the simultaneous extrusion of two materials over stiffening member 326 integrating all three into one proximal shaft 102. Where a high density polyethylene is used for proximal shaft 102 it is preferable that the joint 328 be a polyolefin elastomer or polyolefin polymer with a lower modulus than polyethylene due to their tendency to adhere well to each other. As an alternative to the triple extrusion process, joint 328 may be constructed separately and incorporated into a void left during the manufacture of proximal shaft 102. If less compatible materials are used or if joint 328 is added as a separate unit, it may be necessary to employ an intermediate material to aid adhesion.

Stiffening members 326 may be constructed from metal or polymer and may be formed from wire, plate or rod in a flat, curved or generally cylindrical shape. If stiffening member 326 is curved, it can be pressed into its curved shape, cut from a hypotube, or extruded into a curved shape. If stiffening members 326 are manufactured from metal they may be stainless steel, titanium, tungsten, Nitinol or any other metal known in the art suitable for use in medical devices. It may be preferable, however, to use stainless steel to reduce the cost. If polymeric material is used, they may be any polymeric material having high rigidity and suitable for use in medical devices.

In alternative embodiments of proximal shaft 102, a portion of inflation lumen 118 may include longitudinal stiffness features. As shown in FIGS. 3D and 3E, a lumen stiffening member 330 may be incorporated inside inflation lumen 118. Like stiffening member 326 discussed above, lumen stiffening member 330 may be constructed from metal or polymer and may be formed from wire, plate or rod in a flat, curved or generally cylindrical shape. If stiffening member 330 is curved, it can be pressed into its curved shape, cut from a hypotube, or extruded into a curved shape. If stiffening members 330 are manufactured from metal they may be stainless steel, titanium or any other metal known in the art suitable for use in medical devices. If polymeric material is used, they may be any polymeric material having high rigidity and suitable for use in medical devices.

Further yet, a separate stiffening lumen 332 may be incorporated into proximal shaft 102. FIG. 3F illustrates the use of one such stiffening lumen 332 filled with a biocompatible stiffening fluid 334, such as a saline solution. Stiffening fluid 334 may be sealed in stiffening lumen 332 prior to use or it may be injected into stiffening lumen 332 during use. If stiffening fluid 334 is injected into stiffening lumen 332, the stiffness of the embodiment may be varied by varying the pressure of stiffening fluid 334. A benefit of incorporating a stiffening fluid is that it obviates the need for an additional stiffening component which would make the device both easier and cheaper to construct. In addition, when a balloon catheter is used that employs a stiffening fluid, the same fluid used to inflate the balloon could be used to fill stiffening lumen 332.

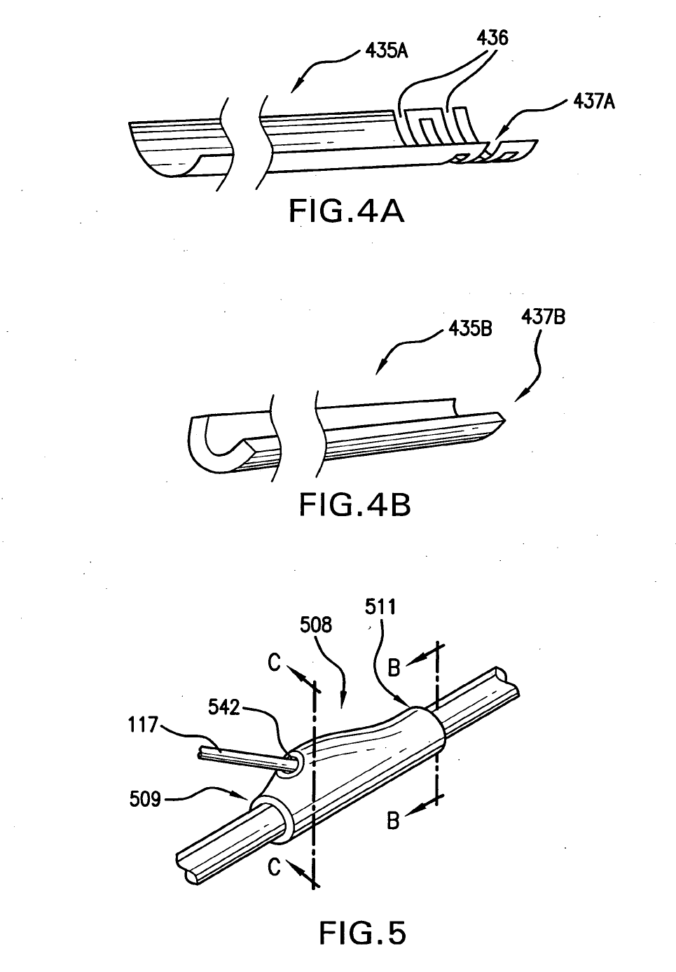

With reference to FIG. 1, multi-lumen catheter 100 may include transition section 104 where the bending stiffness is gradually reduced between a relatively stiff proximal shaft 102 and the relatively flexible distal shaft 106. FIGS. 4A-4B illustrate two embodiments of a transition stiffening member 435A and 435B for use in transition sections 104 where transition section 104 is formed as an integral part of proximal shaft 102. FIG. 4A shows a transition stiffening member 435A which may be incorporated as either a stiffening member 326 or a lumen stiffening member 330. Transition stiffening member 435A has circumferential grooves 436 reducing the stiffness towards its distal end 437A. Similarly, as shown in the embodiment of FIG. 4B, the profile of transition stiffening member 435B may be reduced towards its distal end 437B resulting in a reduction in stiffness. Preferably, transition stiffening member 435B would not be reduced to a point at its distal end 437B. In addition, when a wire is employed, the diameter of the wire may be reduced over a portion of its length to create the stiffness transition. Preferably, the wire diameter would be reduced from approximately 0.017 inch to 0.006 inch.

Guidewire control member 108 allows direct manipulation of guidewire 117 disposed within proximal shaft 102. Direct manipulation of guidewire 117 may be achieved in multiple ways and for multiple purposes, as described below.

FIGS. 5-7 show a guidewire control member 508 according to one embodiment of the present invention. Guidewire control member 508 has proximal and distal ends, 509 and 511 respectively. A catheter receiving bore 640 extends longitudinally through guidewire control member 508 from guidewire control member proximal end 509 to distal end 511. Guidewire control member 508 includes a proximal spreader member 638 and a distal spreader member 639 extending radially into catheter receiving bore 640. The pair of spreader members serve to locally spread open guidewire access cut 110 when guidewire control member 508 is slideably mounted on proximal shaft 102. Guidewire passageway 542 extends through guidewire control member 508 such that its distal most end intersects catheter receiving bore 640 at a shallow angle, preferably ranging from 3 to 15 degrees, between proximal spreader member 638 and distal spreader member 639. As distinguished from proximal spreader member 638, distal spreader member 639 should not project into guidewire lumen 116, where it could interfere with guidewire 117.

Guidewire control member 508 may be molded from a rigid plastic material, such as nylon or nylon based co-polymers, that is preferably lubricous. Alternatively, guidewire control member 508 may be made of a suitable metal, such as stainless steel, or guidewire control member 508 may have both metal components and plastic components. For ease in manufacturing, guidewire control member 508 may be comprised of molded parts that snap-fit together to form the final configuration.

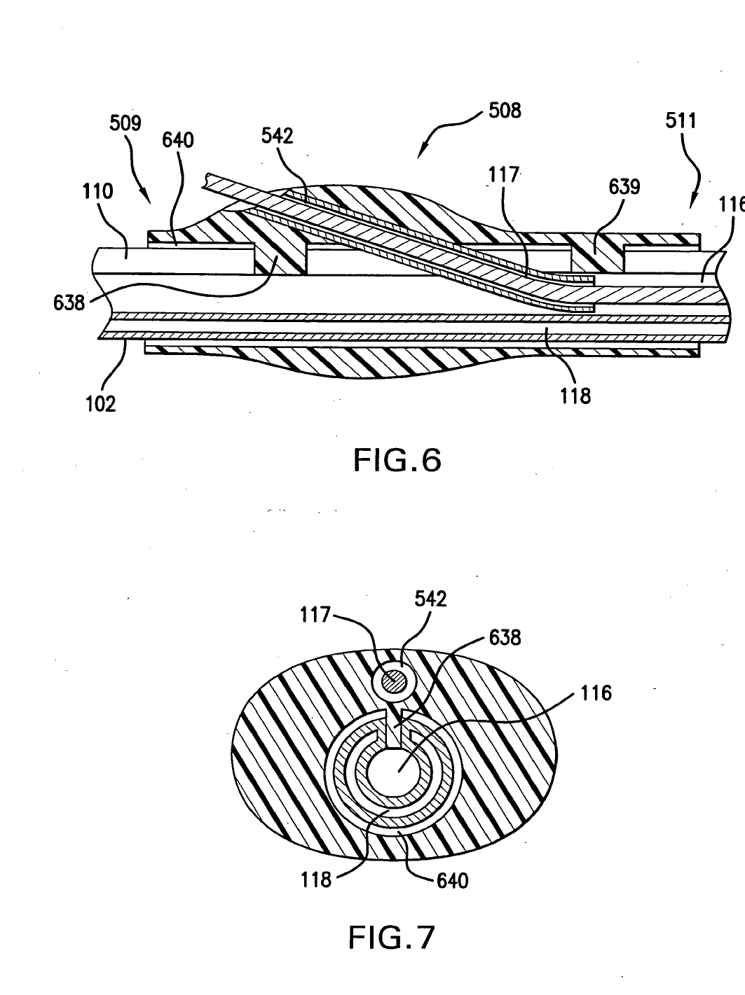

Proximal shaft 102 and guidewire 117 both extend through guidewire control member 508, they merge at the juncture of the passageways, as shown in FIG. 6. Proximal shaft 102 extends through catheter receiving bore 640 of guidewire control member 508, engaging proximal spreader member 638 therein. Proximal spreader member 638 extends through guidewire access cut 110 in proximal shaft 102 to spread guidewire access cut 110 apart as indicated in FIG. 6. Guidewire 117 may extend through guidewire passageway 542 into catheter receiving bore 640 and further into guidewire lumen 116 through the spread open guidewire access cut 110. As proximal shaft 102 is drawn through guidewire control member 508, the once spread open guidewire access cut 110 is drawn closed under the influence of the inherent resiliency of the catheter body, thus enclosing guidewire 117 within guidewire lumen 116.

In an alternative maneuver, guidewire 117 may be inserted or removed through guidewire passageway 542, while guidewire control member 508 is held stationary with respect to multi-lumen catheter 100. In this fashion, guidewire 117 can be removed from multi-lumen catheter 100 and exchanged with another wire. In yet another procedure, guidewire 117 and multi-lumen catheter 100 can be held relatively still while guidewire control member 508 is translated, thus “unzipping” and “zipping” guidewire 117 and proximal shaft 102 transversely apart or together, depending on which direction guidewire control member 508 is moved.

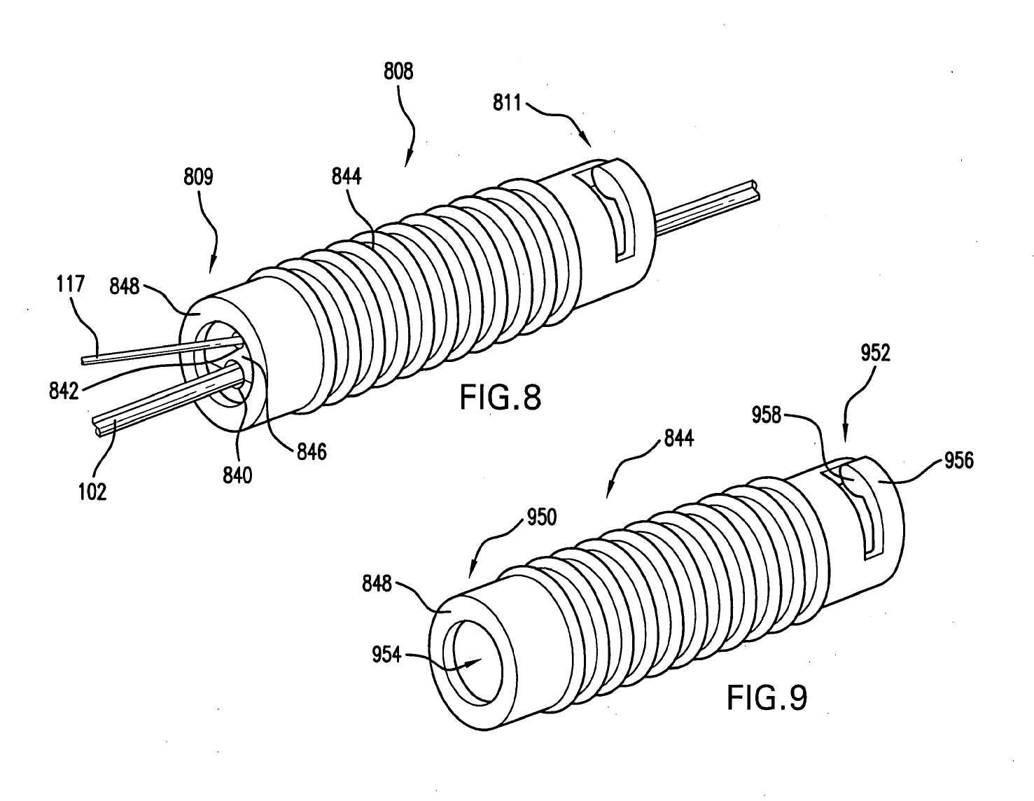

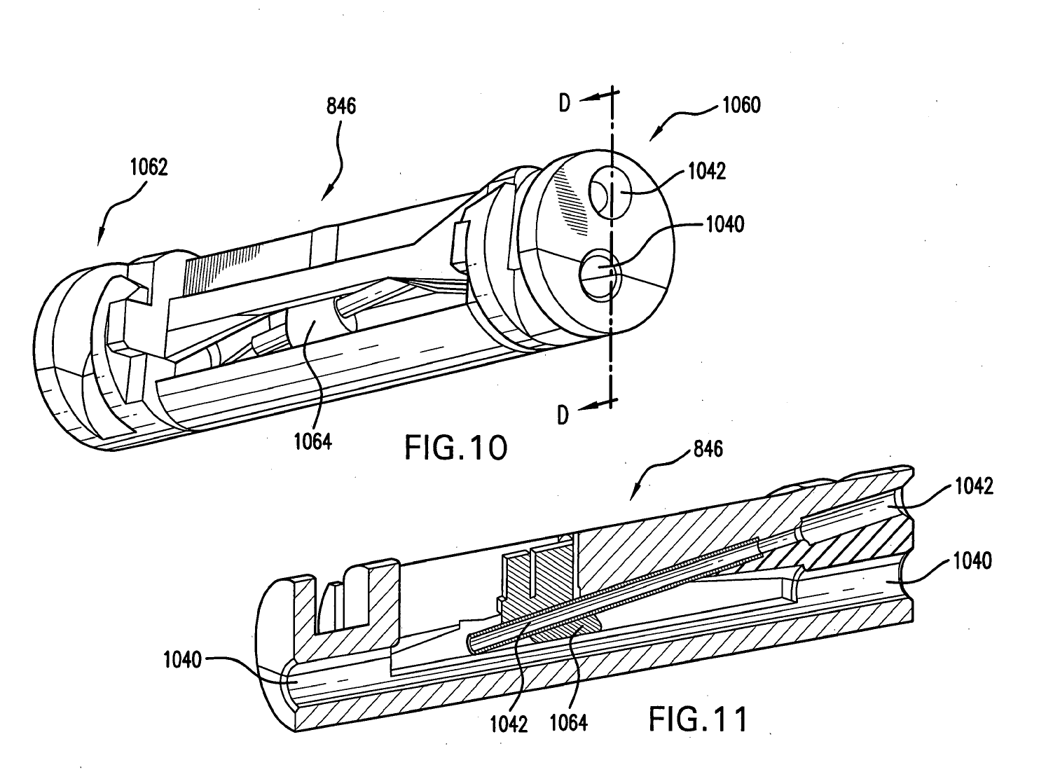

FIGS. 8-11 show an alternate embodiment of a guidewire control member 808. In this instance, guidewire control member 808 surrounds proximal shaft 102 and has a proximal end 809 and a distal end 811. Guidewire control member 808 has an outer tubular member 844 with proximal and distal ends, 950 and 952 respectively, and a longitudinal bore 954 sized to receive an inner body 846. The outer tubular member 844 freely rotates about inner body 846 but is coupled to resist relative axial movement between outer tubular member 844 and inner body 846. A stop shoulder 848 positioned on proximal end 950 of the outer tubular member 844 consists of an annular wall radially extending into the longitudinal bore. The stop shoulder 848 prevents inner body 846 from slipping out of outer tubular member 844 through proximal end 950 of outer tubular member 844.

Two retaining arms 956 are disposed on distal end 952 of outer tubular member 844. Retaining arms 956 consist of two arcuate arms that form a portion of outer tubular member 844. Each arm 956 contains a tab 958 that extends into longitudinal bore 954 of outer tubular member 844 at its distal end 952. When guidewire control member 808 is assembled, the tabs prevent inner body 846 from slipping out of the outer tubular member 844 through its distal end 952. Retaining arms 956 are flexible in the radial direction and may be flexed radially outward to temporarily remove tabs 958 from the longitudinal bore 954 to permit insertion and removal of inner body 846 during the assembly or disassembly of guidewire control member 808. While two tabs 958 are shown positioned 180 degrees apart, a different number of tabs may be used, provided they are spaced sufficiently to prevent inner body 846 from slipping out of the outer tubular member 844. Although the stop shoulder 848 and retaining arms 956 are described as integral parts of the outer tubular member, it should be understood that those features may be created by separate elements such as threaded caps.

Inner body 846, generally functions as the guidewire control member 508, of the previously discussed embodiment. Inner body 846 has proximal and distal ends, 1060 and 1062 respectively. Catheter receiving bore 840 extends longitudinally through inner body 846 from proximal end 1060 to distal end 1062. In the present embodiment, unlike the embodiment shown in FIG. 6, guidewire control member 808 employs a single keel spreader member 1064. Keel spreader member 1064 serves to locally spread open guidewire access cut 110 when guidewire control member 808 is slideably mounted on proximal shaft 102. Guidewire passageway 842 extends through inner body 846 such that its distalmost end intersects catheter receiving bore 840 at a shallow angle, preferably ranging from 3 to 15 degrees. Guidewire passageway 842 extends through keel spreader member 1064 to assure that guidewire 117 travels unobstructed through the spread guidewire access cut 110, as shown in FIG. 11.

It shall be understood that the single keel design may be substituted for the dual spreader design, shown in FIG. 6, and vice versa. In addition, like guidewire control member 508, guidewire control member 808 may be molded from a rigid plastic material, such as nylon or nylon based co-polymers, that is preferably lubricous. Alternatively, guidewire control member 808 may be made of a suitable metal, such as stainless steel, or guidewire control member 808 may have both metal components and plastic components. For ease in manufacturing, guidewire control member 808 may be comprised of molded parts that snap-fit together to form the final configuration.

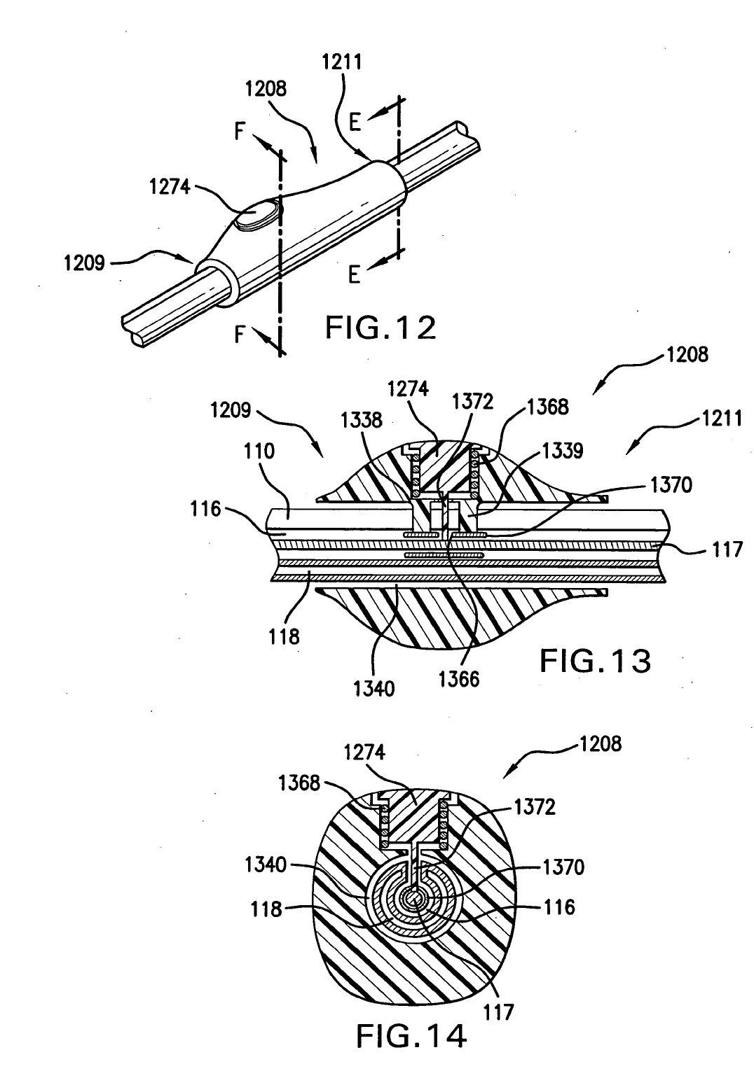

In FIGS. 12-14, a further alternative embodiment of the guidewire control member is illustrated. In this embodiment, guidewire control member 1208 is used to allow direct control over axial movement of indwelling guidewire 117. Such a guidewire control member is disclosed in U.S. Pat. Appl. Publ. No. 2004/0039372 A1, the disclosure of which is incorporated by reference in its entirety herein.

As shown in FIG. 13, guidewire control member 1208 has a main body having both proximal and distal ends, 1209 and 1211 respectively. A catheter receiving bore 1340 extends longitudinally through guidewire control member 1208 from proximal end 1209 to distal end 1211. Guidewire control member 1208 includes a proximal spreader member 1338 and a distal spreader member 1339 extending radially into catheter receiving bore 1340. In addition, a tubular guidewire receiver 1370 is mounted to proximal and distal spreader members, 1338 and 1339 respectively, within catheter receiving bore 1340 and is sized to slideably receive guidewire 117. The pair of spreader members serve to locally spread open guidewire access cut 110 and provide a means for holding tubular guidewire receiver 1370 within guidewire lumen 116 when guidewire control member 1208 is slideably mounted on proximal shaft 102. Tubular guidewire receiver 1370 has a side opening 1366 sized to receive a clamp member 1372. Proximal spreader member 1338 and distal spreader member 1339 serve to align proximal shaft 102 within catheter receiving bore 1340 and especially to align guidewire access cut 110 with side opening 1366 on tubular guidewire receiver 1370.

Clamp member 1372 extends radially inward from a clamp control member 1274. Clamp control member 1274 and clamp member 1372 extend through the guidewire control member 1208 and allow a clinician to manually engage a clamping force on the guidewire 117. In the present embodiment, a clamp spring 1368 is mounted to clamp control member 1274 and guidewire control member 1208. Clamp spring 1368 holds clamp member 1372 and clamp control member 1274 in a disengaged state when no external force is placed on clamp control member 1274. When clamp control member 1274 is pressed and clamp spring 1368 is compressed, it causes clamp member 1372 to extend further radially into the catheter receiving bore 1340, through side opening 1366 in tubular guidewire receiver 1370 and against guidewire 117. That engagement with guidewire 117 results in a frictional force that resists relative movement between guidewire 117 and guidewire control member 1208 allowing a practitioner to directly control the axial location of guidewire 117 within multi-lumen catheter 100.

Like guidewire control members 508 and 808, guidewire control member 1208 may be molded from a rigid plastic material, such as nylon or nylon based co-polymers, that is preferably lubricous. Alternatively, guidewire control member 1208 may be made of a suitable metal, such as stainless steel, or guidewire control ember 1208 may have both metal components and plastic components. For case in manufacturing, guidewire control member 1208 may be comprised of molded parts that snap-fit together to form the final configuration.

While this invention has been particularly shown and described with reference to preferred embodiments thereof, it will be understood by those skilled in the art that various changes in form and details may be made therein without departing from the spirit and scope of the invention.

What is claimed is:

1. A catheter system comprising:
an elongate tubular member having an outer surface and an inner surface defining a wall there between, the tubular member having a generally circular cross-section, and a longitudinal axis;
a guidewire lumen centrally located within and extending along the longitudinal axis of the elongate tubular member and being sized and shaped to slideably receive a guidewire, wherein the guidewire lumen is defined by the inner surface of the elongate tubular member;
a second lumen defined within the wall of the elongate tubular member and having a generally C-shaped cross-section that substantially encircles the guidewire lumen and is concentric therewith, wherein the second lumen has opposing lumen ends defining a web there between, the web being a section of the wall of the elongate tubular member that extends between the outer surface of the elongate tubular member and the guidewire lumen;
a guidewire access cut extending radially through the web from the outer surface of the elongate tubular member to the guidewire lumen to allow transverse access to the guidewire lumen; and
a guidewire control member slideably mounted to the elongate tubular member for accessing the guidewire lumen via the guidewire access cut.

2. The catheter system of claim 1, further comprising:
a balloon mounted on a distal end of the elongate tubular member, wherein the second lumen is an inflation lumen in fluid communication with the balloon.

3. The catheter system of claim 2, further comprising:
at least one stiffening member disposed within the wall of the elongate tubular member between the guidewire lumen and the inflation lumen.

4. The catheter system of claim 2, further comprising:
at least one stiffening member disposed within the wall of the elongate tubular member between the inflation lumen and the outer surface of the tubular member.

5. The catheter system of claim 4, wherein the stiffening member is a metal having a curved shape that substantially encircles the inflation lumen.

6. The catheter system of claim 4, further comprising:
at least one joint disposed within the wall of the elongate tubular member between the second lumen and the outer surface of the tubular member.

7. The catheter system of claim 6, wherein the joint is a groove in the wall of the elongate tubular member.

8. The catheter system of claim 6, wherein the joint is constructed of a polyolefin.

9. The catheter system of claim 2, further comprising:
at least one stiffening member disposed within the inflation lumen.

10. A catheter system comprising:
an elongate tubular member having an outer surface and an inner surface defining a wall there between, the tubular member having a generally circular cross-section;
a guidewire lumen extending longitudinally through the elongate tubular member being sized and shaped to slideably receive a guidewire, wherein the guidewire lumen is defined by the inner surface of the tubular member;
a plurality of inflation lumens defined within the wall of the elongate tubular member and positioned to substantially encircle the guidewire lumen, wherein a web is defined between at least two adjacent inflation lumens, the web extending radially between the outer surface of the elongate tubular member and the guidewire lumen;
at least one stiffening lumen defined within the wall of the elongate tubular member and having a closed distal end, the stiffening lumen extending longitudinally through the elongate tubular member;
a guidewire access cut extending radially through the web from the outer surface of the elongate tubular member to the guidewire lumen to allow transverse access to the guidewire lumen;
a guidewire control member slideably mounted to the elongate tubular member for accessing the guidewire lumen via the guidewire access cut; and
a balloon mounted on a distal end of the elongate tubular member, the balloon being in fluid communication with the plurality of inflation lumens.

11. The catheter system of claim 10, wherein the cross-sectional area of the stiffening lumen is generally constant from a proximal end to the distal end thereof.

12. The catheter system of claim 10, wherein the cross-sectional area of the stiffening lumen is not constant from a proximal end to the distal end thereof.

13. The catheter system of claim 10, further comprising:
a stiffening fluid sealed within the stiffening lumen.

14. The catheter system of claim 13, wherein the stiffening fluid is a saline solution.

15. The catheter system of claim 13, wherein the at least one stiffening lumen is defined within the wall of the tubular member between adjacent inflation lumens.

16. A catheter system comprising:
an elongate tubular member having an outer surface and an inner surface defining a wall therebetween, the tubular member having a generally circular cross-section;
a guidewire lumen extending longitudinally through the elongate tubular member being sized and shaped to slideably receive a guidewire, wherein the guidewire lumen is defined by the inner surface of the tubular member;
a plurality of inflation lumens defined within the wall of the elongate tubular member and positioned to substantially encircle the guidewire lumen, wherein a web is defined between at least two adjacent inflation lumens, the web extending radially between the outer surface of the elongate tubular member and the guidewire lumen;
at least one stiffening member disposed within the wall of the elongate tubular member that extends longitudinally there through;
a guidewire access cut extending radially through the web from the outer surface of the elongate tubular member to the guidewire lumen to allow transverse access to the guidewire lumen;
a guidewire control member slideably mounted to the elongate tubular member for accessing the guidewire lumen via the guidewire access cut; and
a balloon mounted on a distal end of the elongate tubular member, the balloon being in fluid communication with the plurality of inflation lumens.

17. The catheter system of claim 16, wherein the at least one stiffening member is positioned within the wall of the tubular member between adjacent inflation lumens.

18. The catheter system of claim 16, wherein the at least one stiffening member is a metal rod.

19. The catheter system of claim 16, wherein the at least one stiffening member is of a polymeric material.
